(19) United States
(12) Patent Application Publication
(10) Pub. No.: US 2011/0218446 A1
(43) Pub. Date: Sep. 8, 2011

(54) BLOOD PRESSURE MEASURING DEVICE WITH IMPROVED DISPLAY

(75) Inventors: Koji Maruta, Joyo-shi (JP); Tomohiro Kukita, Amsterdam (NL); Kenji Aridome, Osaka (JP); Kohei Takeoka, Kyoto-shi (JP); Yukiko Mitsunami, Otsu-shi (JP)

(73) Assignee: OMRON HEALTHCARE CO., LTD., Kyoto-shi (JP)

(21) Appl. No.: 13/109,663

(22) Filed: May 17, 2011

Related U.S. Application Data
(63) Continuation of application No. PCT/JP2009/068649, filed on Oct. 30, 2009.

(30) Foreign Application Priority Data
Nov. 17, 2008 (JP) 2008-293437

(51) Int. Cl. A61B 5/022

(52) U.S. Cl. 600/490

(57) ABSTRACT

Blood pressure data read from a storage unit is displayed using a blood pressure level bar divided into rectangular segments, and a numerical value of data in a display unit. During the blood pressure measurement, the blood pressure level bar and the numerical value are displayed according to the rise and fall of the detected blood pressure in the display unit. A reference value for determining high blood pressure is displayed in association with the blood pressure level bar. The person to be measured can grasp the blood pressure section to which the measured blood pressure corresponds based on the reference value. The person to be measured can readily grasp the transition of the blood pressure value with the blood pressure level bar even if the size of the display region of the display unit is small.

This is a continuation of application Serial No. PCT/JP2009/068649 filed Oct. 30, 2009, the contents of which are incorporated herein by reference.

### BACKGROUND OF THE INVENTION

1. Field of the Invention

The present invention relates to blood pressure measurement devices, and in particular, to a blood pressure measurement device for displaying a blood pressure measurement result with a bar graph.

2. Description of the Background Art

The conventional electronic sphygmomanometer digitally displays the blood pressure measurement result such as the systolic blood pressure value and the diastolic blood pressure value to notify the measurer (user). The measurer is able to know the past blood pressure value by calling and displaying the history of the blood pressure measurement result. Such display method is effective if the measurer recognizes the measurement result as an accurate value, but the relative high and low levels of the blood pressure value is difficult to determine in a glance. Furthermore, when a plurality of history data is continuously called and displayed to know the transition state of the blood pressure value in time series, the tendency of the blood pressure is difficult to understand instantaneously.

Various devices have been proposed in view of the above problem. For instance, in Japanese Unexamined Patent Publication No. 2004-121632, to which section of the blood pressure section the blood pressure measurement result belongs in a substantially bar shaped region is displayed so as to be readily visualized, but the user cannot know in detail the extent in the section to which the blood pressure measurement result belongs.

In Japanese Unexamined Patent Publication No. 2006-129893, the blood pressure measurement result is displayed as an analog bar graph corresponding to the digital display similar to the conventional mercury sphygmomanometer. The user can readily read the high and low levels of the blood pressure by displaying the positions of the systolic blood pressure and the diastolic blood pressure in the bar graph. In Japanese Unexamined Patent Publication No. 2007-135715, the user can readily read the high and low levels of the blood pressure as the width of the systolic blood pressure and the diastolic blood pressure. However, the display of the determination of the blood pressure section is not made in the devices of such documents, and hence the measurer (user) himself/herself needs to perform the determination based on the blood pressure measurement result.

In Japanese Unexamined Patent Publication No. 61-193643, the stored blood pressure data is calculation processed for every time interval to form a graph, and displayed on a display device. The user then can determine in a glance the transition state of the blood pressure value in time series. However, a display region for displaying the time series data of a constant range is necessary, which enlarges the display device.

### SUMMARY OF THE INVENTION

It is an object of the present invention to provide a blood pressure measurement device having a display mode enabling the level of the blood pressure value, which is difficult to understand in digital display, to be intuitively understood.

Another object of the present invention is to provide a blood pressure measurement device enabling the transition state of the blood pressure value to be determinable in a glance even in a small display region.

In accordance with one aspect of the present invention, there is provided a blood pressure measurement device including: a cuff to be attached to a measurement site of a living body; a control unit for calculating a blood pressure while adjusting pressure of the cuff for blood pressure measurement; a storage unit for storing data of the blood pressure calculated by the control unit; a display unit; and a display operation unit operated to input an instruction related to display using the display unit; wherein the blood pressure indicated by the blood pressure data read from the storage is displayed with a bar divided by a plurality of segments of a predetermined unit and simultaneously displayed with a numerical value in a same screen of the display unit based on the instruction input through the display operation unit.

The blood pressure measurement device preferably includes: a display processing unit for reading out the blood pressure data from the storage unit and displaying the blood pressure indicated by the read blood pressure data with the bar and simultaneously displaying with the numerical value every time a predetermined instruction is input through the display operation unit. The blood pressure data to be read from the storage unit is read according to a time-series order of a measurement time every time the predetermined instruction is input.

The blood pressure data read from the storage unit preferably indicates a systolic blood pressure and a diastolic blood pressure. The systolic blood pressure and the diastolic blood pressure preferably refer to an average of the systolic blood pressures and the diastolic blood pressures of the blood pressure data measured within a predetermined period.

The systolic blood pressure and the diastolic blood pressure preferably refer to an average of the systolic blood pressures and the diastolic blood pressures of the blood pressure data measured within a week.

The systolic blood pressure and the diastolic blood pressure preferably refer to an average of the systolic blood pressures and the diastolic blood pressures of the blood pressure data for a week measured in a predetermined time band of one day.

The predetermined time band preferably refers to a time band corresponding to morning or a time band corresponding to night. A reference value indicating a predetermined blood pressure section is preferably displayed in association at a position indicating a blood pressure value corresponding to the reference value on the bar.

The predetermined blood pressure section preferably refers to a section of early morning high blood pressure. The blood pressure that fluctuates according to adjustment is preferably displayed with a bar and simultaneously displayed with a numerical value in the same screen in the process of adjusting the pressure of the cuff.

Each segment preferably indicates 10 mmHg.

The bar preferably has a square shape.

According to the present invention, the blood pressure indicated by the blood pressure data is displayed with a rectangular bar divided by a plurality of segments of a predetermined unit and simultaneously displayed with a numerical value in the same screen of the display unit. As a result, the user can intuitively understand the level of the blood pressure value that was hard to recognize in the digital display.

The blood pressure data is read out from the storage unit according to the time-series order of the measurement time every time a predetermined instruction is input through the display operation unit. The read blood pressure data is displayed with the rectangular bar and displayed with the numerical value simultaneously on the same screen every time the data is read out. The user then can determine the transition state of the blood pressure value according to time series in a glance even in a small display region such as a screen.

The foregoing and other objects, features, aspects and advantages of the present invention will become more apparent from the following detailed description of the present invention when taken in conjunction with the accompanying drawings.

### BRIEF DESCRIPTION OF THE DRAWINGS

FIG. 1 is a schematic view of an automatic winding electronic sphygmomanometer according to an embodiment.

FIG. 2 is a schematic configuration diagram of an air system of the automatic winding electronic sphygmomanometer according to the embodiment.

FIG. 3 is a view schematically showing a usage mode at the time of blood pressure measurement of the automatic winding electronic sphygmomanometer according to the embodiment.

FIG. 4 is a hardware configuration diagram of the automatic winding electronic sphygmomanometer according to the embodiment.

FIG. 5 is a function configuration diagram of the automatic winding electronic sphygmomanometer according to the embodiment.

FIG. 6 is a memory configuration diagram of the automatic winding electronic sphygmomanometer according to the embodiment.

FIG. 7 is a flowchart of the blood pressure measurement process according to the embodiment.

FIG. 8 is a flowchart of a calling and displaying process according to the embodiment.

FIG. 9 is a view showing one example of a display according to the embodiment.

FIG. 10 is a view showing another example of a display according to the embodiment.

FIG. 11 is a view showing another further example of a display according to the embodiment.

FIG. 12 is a view showing another further example of a display according to the embodiment.

FIG. 13 is a view showing another further example of a display according to the embodiment.

FIG. 14 is a schematic view of another blood pressure measurement device according to the embodiment.

### DESCRIPTION OF THE PREFERRED EMBODIMENTS

Each embodiment of the present invention will be described in detail with reference to the drawings. The same reference numerals are denoted for the same or corresponding portions throughout the drawings, and the description thereof will not be repeated.

(Automatic Winding Electronic Sphygmomanometer)

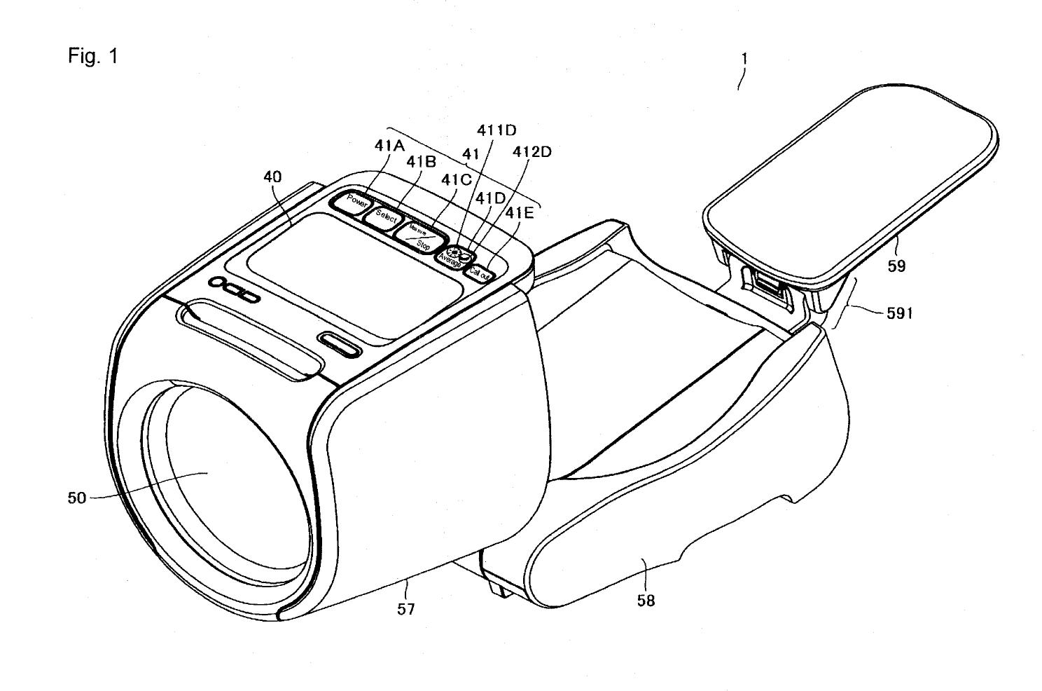

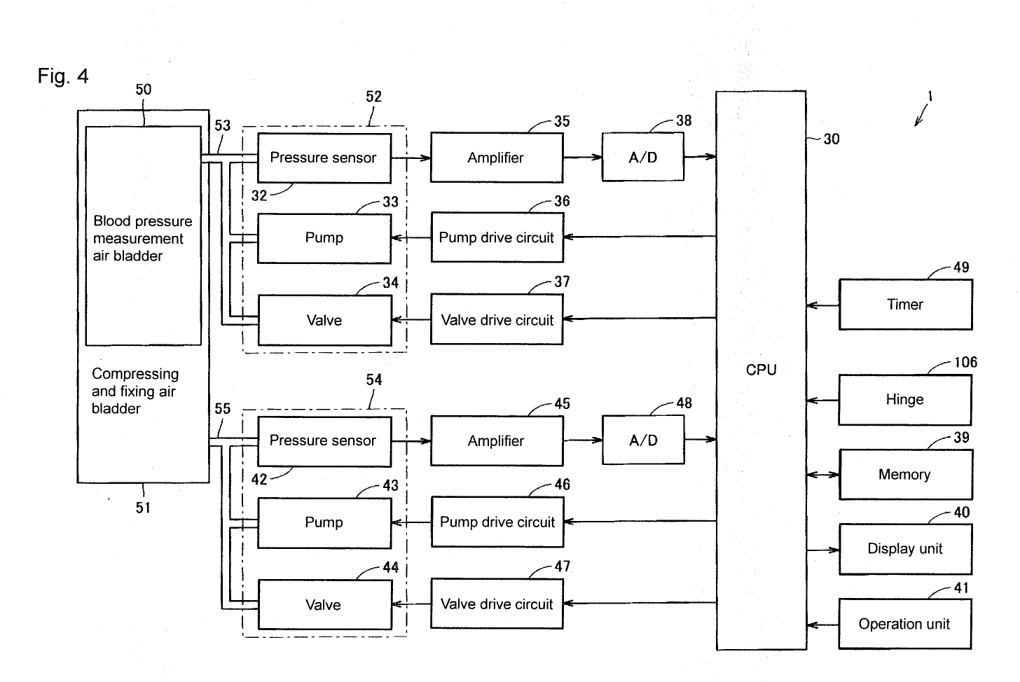

An automatic winding electronic sphygmomanometer 1 having the configuration of FIG. 1 to FIG. 4 is illustrated as a blood pressure measurement device according to the present invention.

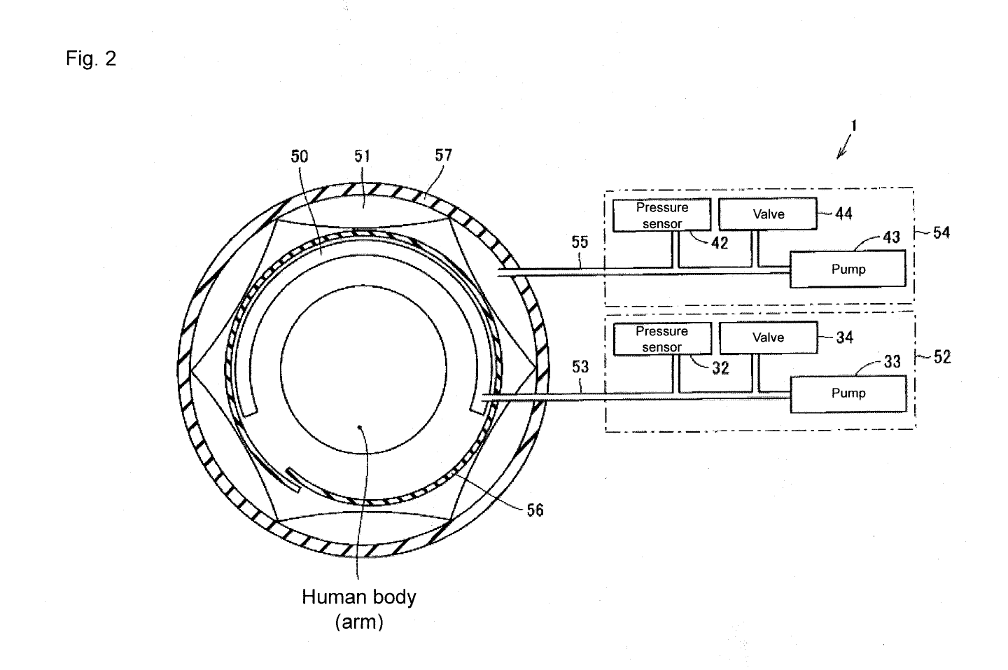

With reference to FIG. 2 to FIG. 4, the automatic winding electronic sphygmomanometer 1 includes a blood pressure measurement air bladder 50, a compressing and fixing air bladder 51 for compressing the blood pressure measurement air bladder 50 and fixing the same at the measurement site, a blood pressure measurement air system 52 for supplying or discharging air to and from the blood pressure measurement air bladder 50 through a tube 53, and an amplifier 35, a pump drive circuit 36, a valve drive circuit 37, and an A/D (Analog/Digital) converter 38 arranged in relation to the blood pressure measurement air system 52. The automatic winding electronic sphygmomanometer 1 also includes a compressing and fixing air system 54 for supplying or discharging air to and from the compressing and fixing air bladder 51 through a tube 55, and an amplifier 45, a pump drive circuit 46, a valve drive circuit 47, and an A/D converter 48 arranged in relation to the compressing and fixing air system 54. The automatic winding electronic sphygmomanometer 1 further includes a CPU (Central Processing Unit) 30 for intensively controlling and monitoring each unit, a memory 39 for storing various types of information such as the measured blood pressure value, a display unit 40 for displaying various types of information including the blood pressure measurement result, an operation unit 41 operated to input various types of instructions for measurement, a timer 49 for timing the current time and a hinge 106 incorporating a sensor 107 to be described later.

The blood pressure measurement air system 52 includes a pressure sensor 32 for detecting and outputting the pressure (hereinafter referred to as cuff pressure) in the blood pressure measurement air bladder 50 (correspond to the cuff to be described later), a pump 33 for supplying air to the blood pressure measurement air bladder 50, and a valve 34 to be opened and closed to exhaust or enclose the air of the blood pressure measurement air bladder 50. The amplifier 35 receives the output signal of the pressure sensor 32, amplifies such input signal and provides the amplified signal to the A/D converter 38. The A/D converter 38 receives the provided analog signal, converts such input signal to a digital signal, and outputs to the CPU 30. The pump drive circuit 36 controls the drive of the pump 33 based on a control signal provided from the CPU 30. The valve drive circuit 37 controls the opening and the closing of the valve 34 based on a control signal provided from the CPU 30.

The compressing and fixing air system 54 includes a pressure sensor 42 for detecting and outputting the pressure in the compressing and fixing air bladder 51, a pump 43 for supplying air to the compressing and fixing air bladder 51, and a valve 44 to be opened and closed to exhaust or enclose the air of the compressing and fixing air bladder 51. The amplifier 45 receives the output signal of the pressure sensor 42, amplifies such input signal and provides the amplified signal to the A/D converter 48. The A/D converter 48 receives the provided analog signal, converts such input signal to a digital signal, and outputs to the CPU 30. The pump drive circuit 46 controls the drive of the pump 43 based on a control signal provided from the CPU 30. The valve drive circuit 47 controls the opening and the closing of the valve 44 based on a control signal provided from the CPU 30.

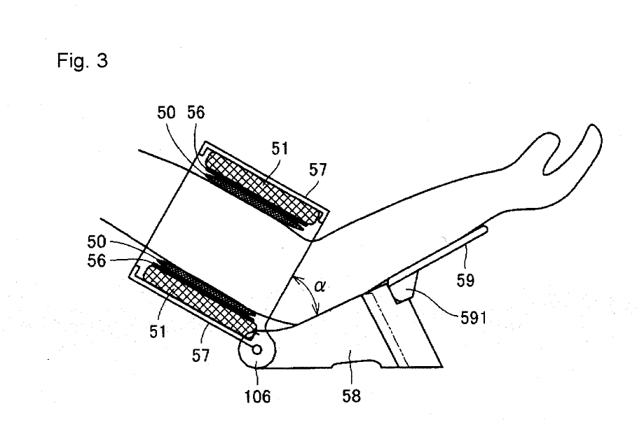

With reference to FIG. 1 and FIG. 3, the automatic winding electronic sphygmomanometer 1 includes a fixing tubular case 57 for fixing the upper arm, which is a measurement site of the person to be measured, a sphygmomanometer main body 58, and a mounting portion 59 for placing the arm at the elbow joint or lower at the time of measurement. The fixing tubular case 57 includes the display unit 40 such as the LCD (Liquid Crystal Display), and the operation unit 41 arranged so that the person to be measured can operate from the outside.

The operation unit 41 includes a power switch 41A, a switch 41B operated to select the person to be measured, a switch 41C for instructing start and stop of the blood pressure measurement, and switches 41D and 41E operated to read out the stored measurement data and display on the display unit 40. A series of operations from reading out the measurement data from the storage unit and displaying the read measurement data is referred to as the calling and displaying herein. The switch 41D includes a switch 411D operated to call and display the morning time measurement data, and a switch 412D operated to call and display the night time measurement data.

In the present embodiment, it is assumed that the automatic winding electronic sphygmomanometer 1 can store the blood pressure measurement data for two people. The users A and B can be specified by operating the switch 41B. A visitor other than the users A and B can also be specified by operating the switch 41B. If the visitor is specified, the blood pressure measurement is performed but the blood pressure measurement data is not stored. The automatic winding electronic sphygmomanometer 1 can store the blood pressure measurement data for two people but may be able to store the data for three or more people.

The fixing tubular case 57 includes the blood pressure measurement air bladder 50 to be attached to the measurement site at the inner circumferential surface. FIG. 3 shows a state in which the upper arm that is the measurement site of the person to be measured is inserted from the nearside direction in the drawing of the fixing tubular case 57 and fixed thereat.

The automatic winding electronic sphygmomanometer 1 has the mounting portion 59 folded towards the sphygmomanometer main body 58 side through a connecting portion 591, and the fixing tubular case 57 folded towards the sphygmomanometer main body 58 side through the hinge 106 to take an integrated configuration when not in use. At the time of blood pressure measurement and when reading out and displaying the stored measurement data, the person to be measured turns the fixing tubular case 57 towards the nearside direction (person to be measured side) in the drawing through the hinge 106 as shown in FIG. 1 from the integrated configuration state to separate from the sphygmomanometer main body 58. The person to be measured then can insert the arm to the fixing tubular case 57, as shown in FIG. 3. In such state, the fixing tubular case 57 and the sphygmomanometer main body 58 are connected through the hinge 106.

FIG. 2 schematically shows the transverse cross-section of the fixing tubular case 57 in the state of FIG. 3. The blood pressure measurement air bladder 50, a compressing and fixing curler 56, and the compressing and fixing air bladder 51 are arranged so as to be able to be wrapped around the upper arm in the fixing tubular case 57 from the outer periphery of the upper arm that is the measurement site towards the inner circumferential surface of the fixing tubular case 57. The compressing and fixing curler 56 is wrapped around the upper arm. The shape of the compressing and fixing curler 56 when wrapped around is substantially circular that lies along the periphery of the upper arm. The diameter of the substantially circular shape is freely extendable. The diameter of the compressing and fixing curler 56 is reduced when the air is gradually supplied by the compressing and fixing air system 54 thereby expanding the compressing and fixing air bladder 51, so that the blood pressure measurement air bladder 50 interposed between the compressing and fixing curler 56 and the human body (upper arm) is pressed against the measurement site. The blood pressure measurement air bladder 50 is wrapped around and fixed to the periphery of the human body (arm) by the compressing and fixing curler 56 and the compressing and fixing air bladder 51, so that the blood pressure can be measured.

(Regarding Function Configuration)

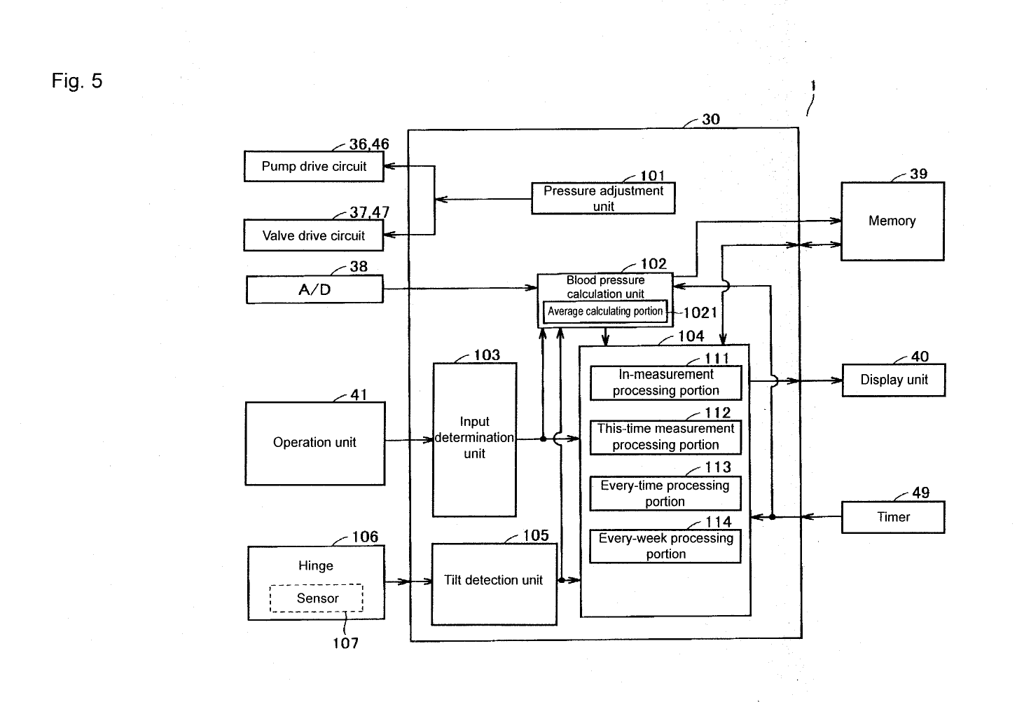

The function configuration of the automatic winding electronic sphygmomanometer 1 according to the present embodiment will be described with reference to FIG. 5. The automatic winding electronic sphygmomanometer 1 includes a pressure adjustment unit 101, a blood pressure calculation unit 102 including an average calculating portion 1021, an input determination unit 103, a display processing unit 104, and a tilt detection unit 105. The pressure adjustment unit 101 adjusts the inner pressure of the blood pressure measurement air bladder 50 and the compressing and fixing air bladder 51 by controlling the pump drive circuits 36 and 46, and the valve drive circuits 37 and 47.

The blood pressure calculation unit 102 calculates the blood pressure based on the signal input from the A/D converter 38, and stores the calculation result in the memory 39. The calculation result is output to the display processing unit 104 for display. The details on the functions of the blood pressure calculation unit 102 will be hereinafter described.

When detecting that the time data indicates a predetermined day of the week (e.g., Sunday) of every week based on the time data timed by the timer 49, the average calculating portion 1021 reads out the blood pressure measurement data for the past one week from the memory 39, calculates the average measurement value based on the read measurement data for one week, and stores the calculated average data in the memory 39. The details of the functions of the average calculating portion 1021 will be described later.

The input determination unit 103 inputs the signal output when the operation unit 41 is operated by the person to be measured, determines which switch of the operation unit 41 is operated based on the input signal, and outputs the determination result. Specifically, the input determination unit 103 stores in advance the signal level output when the switch is operated in correspondence for every type of switch. When the user operates the switch, the level of the signal input from the operation unit 41 and the stored level are compared and matched, and the type of switch to store in correspondence with the matching level is specified. The type of operated switch then can be determined.

The tilt detection unit 105 is arranged in association with the sensor 107 of the hinge 106. The sensor 107 detects the tilt angle (see angle α of FIG. 3) of the fixing tubular case 57 with respect to the sphygmomanometer main body 58 through the hinge 106. The signal of the detected tilt angle is provided to the tilt detection unit 105. The tilt detection unit 105 compares the angle indicated by the input tilt angle signal and a predetermined angle stored in advance, and outputs the signal based on the comparison result to the display processing unit 104 and the blood pressure calculation unit 102 as a tilt detection signal.

The display processing unit 104 has a function of displaying data on the display unit 40. Specifically, the display processing unit 104 includes an in-measurement processing portion 111 for performing a display of in-blood pressure measurement, a this-time measurement processing portion 112 for displaying the blood pressure measurement result for this time at the end of the blood pressure measurement, an every-time processing portion 113 for reading out and displaying the measurement result for each time stored in the memory 39, and an every-week processing portion 114 for reading out and displaying the average blood pressure measurement result in units of weeks stored in the memory 39.

The functions of the pressure adjustment unit 101, the blood pressure calculation unit 102, the input determination unit 103, the display processing unit 104, and the tilt detection unit 105 are stored in the memory 39 as programs in advance. The CPU 30 reads out such programs from the memory 39, and executes the read programs to realize the corresponding function of each unit.

In FIG. 5, only the circuits associated with the functions executed by the CPU 30 are shown as peripheral circuits that perform input and output with the CPU 30.

(Regarding Memory Configuration)

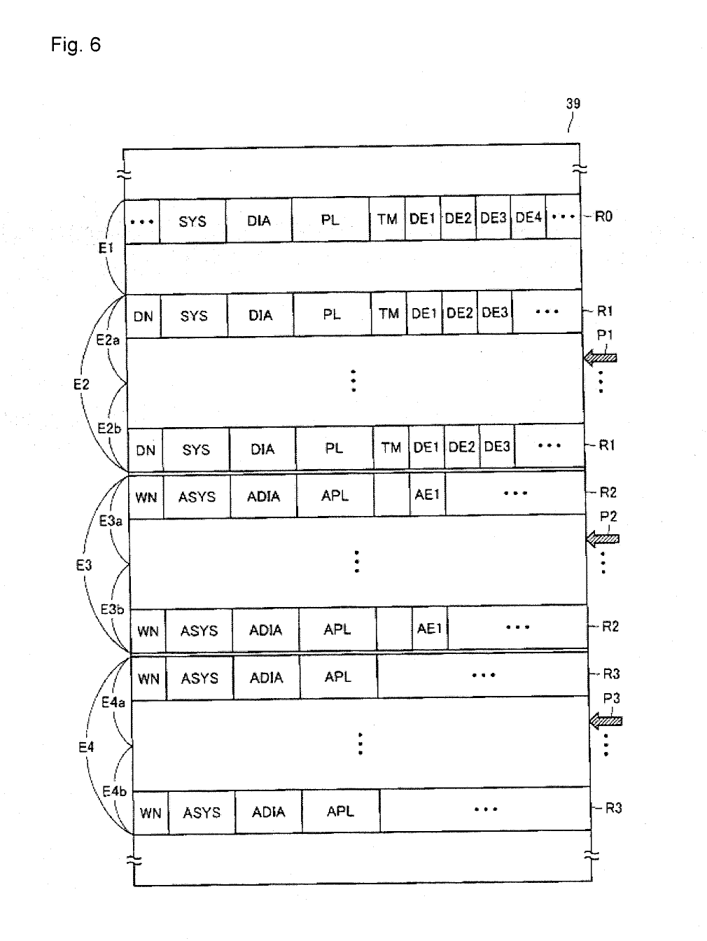

FIG. 6 shows one example of a storage content of the memory 39. The memory 39 includes regions E1, E2, E3, and E4. The region E1 is a region where the measurement result is temporarily stored in the form of a record R0 when the blood pressure measurement is performed. Each region E2, E3, and E4 includes a region for storing the blood pressure measurement result for each user A and B.

The record R0 of the region E1 that is the blood pressure measurement result is read every time the blood pressure measurement is performed, and the read record R0 is stored in the region E2 in the form of a record R1. More specifically, the record R1 of the blood pressure measurement result of the user A is stored in a region E2a of the region E2, and the record R1 of the blood pressure measurement result of the user B is stored in a region E2b of the region E2.

In the region E3, the average data of the morning time measurement data calculated every week based on the blood pressure measurement result stored in the region E2 is stored in the form of a record R2. More specifically, the average data of the morning time measurement data in units of weeks based on the data stored in the region E2a of the user A is stored in a region E3a. Similarly, the average data of the morning time measurement data in units of weeks based on the data stored in the region E2b of the user B is stored in a region E3b of the region E3.

In the region E4, the average data of the night time measurement data calculated every week based on the blood pressure measurement result stored in the region E2 is stored in the form of a record R3. More specifically, the average data of the night time measurement data in units of weeks based on the data stored in the region E2a of the user A is stored in a region E4a of the region E4. Similarly, the average data of the night time measurement data in units of weeks based on the data stored in the region E2b of the user B is stored in a region E4b of the region E4.

The record R0 of the region E1 includes, for the blood pressure measurement result of this time, systolic blood pressure data SYS, diastolic blood pressure data DIA, pulse rate data PL, measurement time data TM, data DE1 indicating whether corresponding to early morning high blood pressure, data DE2 indicating whether the person to be measured moved his/her body during the blood pressure measurement, data DE3 indicating whether or not the tilt angle of the fixing tubular case 57 is shifted from a predetermined angle for normal measurement, that is, tilted, and data DE4 for identifying the user.

The data TM refers to the measurement time. The blood pressure calculation unit 102 stores the measurement data input from the timer 49 in the record R0 as the data TM.

The data DE4 indicates a person different from the person to be measured indicated by the operation of the switch 41B. The signal the blood pressure calculation unit 102 inputs from the input determination unit 103 is stored in the record R0 as the data DE4.

The data DE3 indicates the detection result on whether or not the angle α of the fixing tubular case 57 detected by the sensor 107 during the blood pressure measurement is deviated from a range of a predetermined angle by the tilt detection unit 105. The range of the predetermined angle is the range of the angle α detected when the measurer is in a normal measurement position. The data of the range of the predetermined angle is assumed to be detected by experiment or the like in advance, and stored in a predetermined storage region of the memory 39.

The data DE2 indicates the result of detecting whether or not the person to be measured moved his/her body during the blood pressure measurement. If the person to be measured moved his/her body during the measurement, the measurement accuracy is known to lower. The CPU 30 can detect the presence of body motion based on the waveform of the pulse wave detected during the blood pressure measurement. The details will be omitted herein since the well known technique can be applied to the procedure for detecting the body motion. The determination result of the angle α of the fixing tubular case 57 by the tilt detection unit 105 and the detection result on the presence of the body motion are output to the blood pressure calculation unit 102. The blood pressure calculation unit 102 stores the detection result input from the tilt detection unit 105 and the detection result of the body motion in the record R0 as the data DE2 and DE3, respectively.

The record R1 stored in the region E2a includes data DN indicating the serial number complying with the order stored in the region E2a, that is, the order of measurement time, the data SYS, DIA, PL and TM, as well as data DE1, DE2 and DE3. The record R1 is similarly stored in the region E2b for the user B. A maximum of 99 records R1 can be stored in each region E2a and E2b.

The record R2 of the region E3a includes data representing the average of the measurement data in units of one week calculated based on the record R1 in which the data TM indicates the morning time of the records R1 stored in the region E2a. The record R2 includes data WN indicating which week of data, data ASYS indicating the average of the systolic blood pressure data SYS of the morning time measurement for one week, data ADIA indicating the average of the diastolic blood pressure data DIA of the morning time measurement for one week, data APL indicating the average of the data of the pulse rate of the morning time measurement for one week, and data AE1 indicating whether or not the average blood pressure indicated by the data SYS and ADIA stored in the record R2 indicates the early morning high blood pressure. Similarly, the record R2 of the region E3b includes the data ASYS, ADIA, APL, and AE1 calculated in units of one week based on the data stored in the region E2b.

The record R3 of the region E4a includes data of an average value in units of one week of the night time measurement data based on the record R1 in which the data TM indicates the night time of the records R1 stored in the region E2a. Similarly, the record R3 of the region E4b includes the data of the average value in units of one week of the night time measurement data based on the record R1 in which the data TM indicates the night time of the records R1 stored in the region E2b.

The record for a maximum of eight weeks is stored in each region E3a, E3b, E4a, and E4b. Specifically, the records for a total of eight weeks, for this week (data WN indicates “0”), last week (one week before; data WN indicates “1”, week before (two weeks before; data WN indicates “2”). If the data for this week is newly calculated, the newly calculated content is overwritten on the oldest stored record, that is, the record in which the value indicated by the data WN is “7”. When overwrite is performed, the value of the data WN of the record is updated to “7” to “0”, and the value of the data WN of each record is updated by +1.

The pointers P1, P2, and P3 are arranged in each region E2, E3, and E4. The pointer P1 indicates the record R1 in which the data is currently read out in the region E2. The pointer P2 indicates the record R2 in which the data is currently read out in the region E3. The pointer P3 indicates the record R3 in which the data is currently read out in the region E4.

(Regarding Blood Pressure Calculation/Average Calculation Function)

The blood pressure calculation unit 102 calculates the blood pressure (highest blood pressure (systolic blood pressure) and the lowest blood pressure (diastolic blood pressure)) according to a well known method such as the oscillometric method based on the pulse wave signal input from the A/D converter 38. The pulse is also calculated by the well known method.

Furthermore, the blood pressure calculation unit 102 determines whether or not the blood pressure corresponds to the early morning high blood pressure, the presence of body motion during the measurement, whether or not the tilt angle α of the fixing tubular case 57 during the measurement is appropriate, and the user every time the blood pressure measurement is carried out, and stores the determination result in the memory 39 in association with the measurement data.

According to the Japanese Society of Hypertension, high blood pressure is determined when the systolic blood pressure is higher than or equal to 135 mmHg or the diastolic blood pressure is higher than or equal to 85 mmHg in the home blood pressure. In particular, the early morning high blood pressure is determined when the blood pressure after waking up corresponds to such conditions of high blood pressure. The early morning high blood pressure may become the cause of cardiovascular risk. In the present embodiment, therefore, determination is made as the morning time measurement if detected that the blood pressure measurement is carried out between 4 AM and 10 AM in one day and determination is made as the night time measurement if detected that the blood pressure measurement is carried out between 7 PM and 2 AM based on the timing data of the timer 49. Furthermore, the morning time measurement data SYS and DIA and the high blood pressure index data (135 mmHg/85 mmHg) are compared to detect whether or not the measured blood pressure is the early morning high blood pressure based on the comparison result. The detection result is stored as the data DE1.

The data indicating morning/night time (4 AM to 10 AM/7 PM to 2AM) and the high blood pressure index data (135 mmHg/85 mmHg) are stored in a predetermined storage region of the memory 39 in advance.

The average calculating portion 1021 receives the signal generated by the operation of the switch 41B through the input determination unit 103, and identifies the user based on the input signal. The past measurement data stored in the region of the memory 39 corresponding to the identified user is then searched, and the morning time measurement data for one week is read out. The average data of the read data is calculated. Similarly, the average data of the night time measurement data for one week is calculated.

Specifically, if determined that the timing data of the timer 49 indicates a predetermined day of the week (e.g., Sunday), the measurement data (measurement data of record R1 in which data TM indicates 4 AM to 10 AM) of the record R1 of the morning time for the past one week is read from the region E2 of the memory 39 for every user, the average of the read measurement data for one week is calculated, and the calculated result is stored in the region E3 of the memory 39. Similarly, the night time measurement data (measurement data of record R1 in which data TM indicates 7 PM to 2 AM) for the past one week is read from the region E2 of the memory 39 for every user, the average of the read measurement data for one week is calculated, and the calculated result is stored in the region E4 of the memory 39.

The average calculating portion 1021 compares the calculated morning time average measurement data ASYS and ADIA and the high blood pressure index data (135 mmHg/85 mmHg), and detects whether or not the early morning high blood pressure based on the comparison result. The detection result is stored in the record R2 as the data AE1.

The average calculating portion 1021 calculates the average of the morning time and night time measurement data for this week based on the measurement data of the record R1 of the morning time and the night time for this week (from Sunday immediately before until today) of the region E2 every day for every user every time the blood pressure measurement is carried out, and stores the result in the regions E3 and E4 of the memory 39 as the records R2 and R3 (data WN is “0”).

(Blood Pressure Measurement Process)

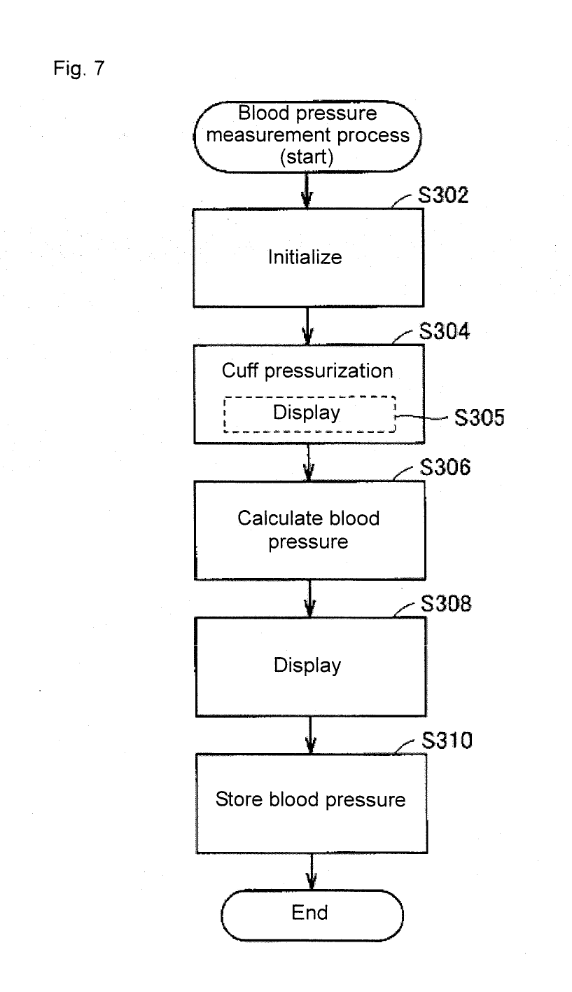

The blood pressure measurement process according to the present embodiment will be described according to the flowchart of FIG. 7. The blood pressure measurement process described below is an example, and the blood pressure measurement method is not particularly limited.

In the measurement state shown in FIG. 3, the CPU 30 first performs an initialization process (step S302). Specifically, the exhaust of air of the blood pressure measurement air bladder 50 and the compressing and fixing air bladder 51, the correction of the pressure sensors 32 and 34, and the like are carried out.

In the measureable state, the pressure adjustment unit 101 drives the pump drive circuits 36 and 46 according to a predetermined procedure, and gradually raises the pressure of the blood pressure measurement air bladder 50 and the compressing and fixing air bladder 51 (step S304). When the blood pressure measurement air bladder 50 is wrapped around and fixed to the measurement site by the compressing and fixing air bladder 51, the inner pressure (cuff pressure) of the blood pressure measurement air bladder 50 gradually rises, and the pressure adjustment unit 101 controls the pump drive circuit 36 to stop the pump 33 when a predetermined level for blood pressure measurement is reached. In the pressurization process, a display process using the display unit 40 by the in-measurement processing portion 111 is carried out (step S305). In the present embodiment, the blood pressure calculation is carried out in the pressurization process from when the cuff pressure rises to when a predetermined level is indicated.

In the pressurization process, the pulse pressure signal superimposed on the cuff pressure signal detected by the pressure sensor 32 through A/D converter 38 is provided to the blood pressure calculation unit 102. The blood pressure calculation unit 102 converts the input pulse pressure signal to the blood pressure data, and sequentially outputs the blood pressure data obtained by conversion to the in-measurement processing portion 111. The in-measurement processing portion 111 generates the image data based on the input data, and outputs the generated image data to the display unit 40. The display unit 40 displays the image based on the input image data.

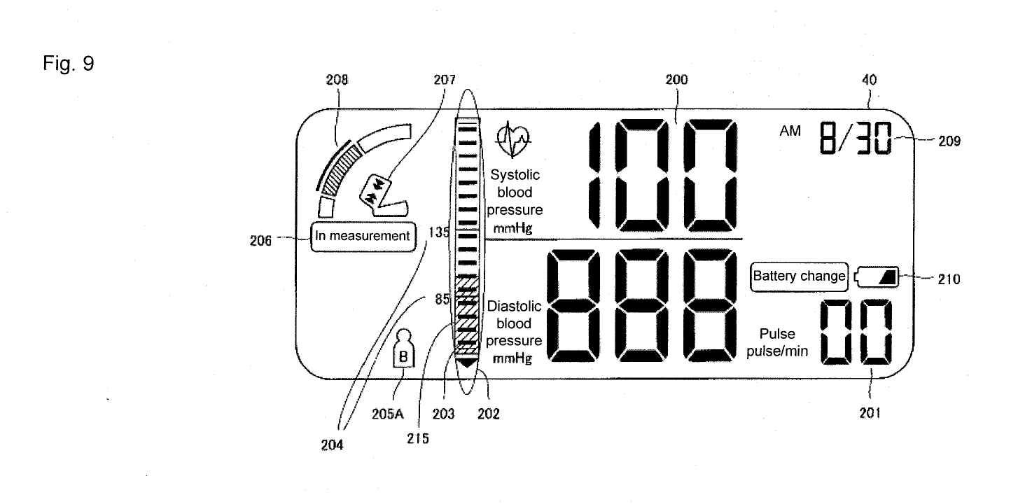

One example of the display screen in the pressurization process is shown in FIG. 9. With reference to FIG. 9, data 200 showing the current blood pressure, pulse rate data 201, blood pressure level bar 202, data 205A showing another user, data 206 showing that measurement is being carried out, data 207 showing that pressurization is being carried out, data showing the extent of tilt of the fixing tubular case 57 based on the detection signal by the tilt detection unit 105, current time data 209 indicated by the timer 49, battery change alarm 210, and reference value 204 that is an index of the early morning high blood pressure shown in association with the blood pressure level bar 202 are simultaneously displayed on the display unit 40 in the same screen. In FIG. 9, “00” is displayed for the value of the pulse rate data 201 since the pulse rate is not calculated.

In FIG. 9, the reference value 204 for determining the early morning high blood pressure section is displayed in association at a position indicating the blood pressure value corresponding to the reference value 204 on the blood pressure level bar 202. The reference value 204 is displayed herein, but may be printed in advance on the screen of the display unit 40.

In FIG. 9, a bar graph 215 with a shaded area showing the value of the blood pressure indicated by the data 200 is simultaneously displayed superimposed on the blood pressure level bar 202. The length of the bar of the bar graph 215 extends and contracts in cooperation with the change in value of the data 200 so as to indicate the value of the data 200. A rectangular mark 203 is displayed as a scale for sectionalizing into segments of 10 mmHg units on the blood pressure level bar 202 so that the value indicated by the bar graph 215 can be readily read. The mark 203 is transparently displayed through the bar graph 215. Therefore, the bar graph 215 on the blood pressure level bar 202 of square, preferably rectangular shape and the digital numerical value by the data 200 are simultaneously displayed, and the bar of the bar graph 215 extends and contracts in cooperation with the change in value of the data 200 in the same screen of the display unit 40, so that the person to be measured can intuitively check the progress status of pressurization with the display screen of FIG. 9.

In the pressurization process, the blood pressure calculation unit 102 calculates the blood pressure (systolic blood pressure, diastolic blood pressure) according to a known procedure based on the pressure pulse wave signal detected through the AID converter 38 (step S306). The blood pressure calculation unit 102 calculates the pulse rate with a known procedure.

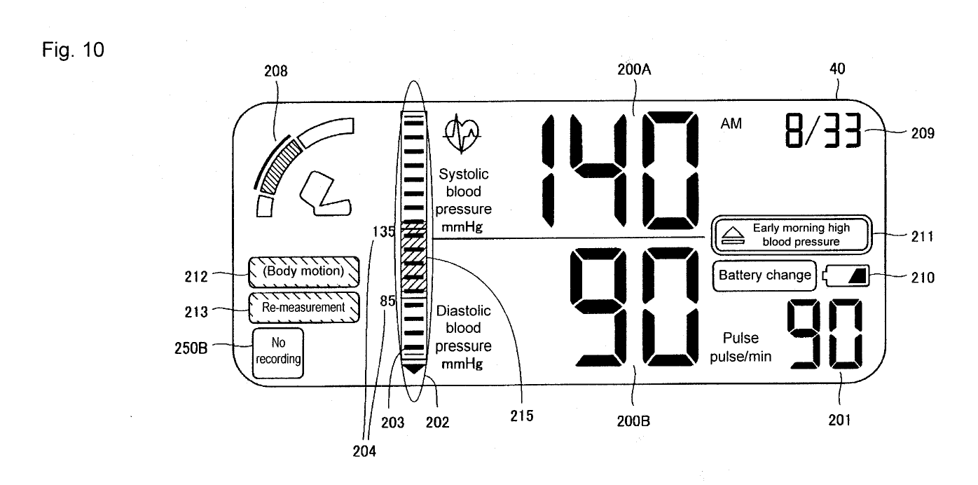

The calculated blood pressure and the pulse rate are stored in the region E1 of the memory 39 as record R1, and the content of the record R0 is provided to the this-time measurement processing portion 112 of the display processing unit 104. The this-time measurement processing portion 112 generates image data based on the provided data, and outputs to the display unit 40 (step S308). The display unit 40 displays the screen of FIG. 10, for example, based on the input image data.

In the screen of FIG. 10, values 200A and 200B of the systolic blood pressure and the diastolic blood pressure according to the data SYS and DIA of the record R0, and data 211, 212, and 213 based on the data DE1, DE2, and DE3 of the record R0 are displayed. If body motion is detected during the measurement (if data DE2 is displayed), determination is made that the measurement accuracy is low. The alarm data 213 urging “re-measurement” is thus displayed on the screen.

If the person to be measured does not operate the switch 41B when carrying out the blood pressure measurement, that is if the user A or B is not selected, the blood pressure measurement result is not stored in the region E2 or E3 of the memory 39 in the present embodiment. If the blood pressure measurement is carried out with the person to be measured not operating the switch 41B, data 250B informing the person to be measured that the measurement data will “not be recorded” is displayed in the display unit 40.

In FIG. 10, the bar graph 215 with a shaded area is displayed superimposed on the blood pressure level bar 202. The values on the blood pressure level bar 202 corresponding to the systolic blood pressure and the diastolic blood pressure indicated by the data 200A and 200B are indicated by the upper end and the lower end, respectively, of the bar of the bar graph 215. Similar to FIG. 9, the mark 203 of the blood pressure level bar 202 is transparently displayed through the bar graph 215 so that the person to be measured can readily read the blood pressure value.

Following the display process of step S308, the blood pressure calculation unit 102 generates the record R1 based on the data of the record R0, and stores the generated record R1 in the region for the relevant user of the region E2 of the memory 39 (step S310). Assume here that the person to be measured is selected in advance by the operation of the switch 41B as user A or B, so that the generated record R1 is stored in the region E2a or E2b based on the operation signal of the switch 41B.

After the blood pressure measurement, the air of the blood pressure measurement air bladder 50 and the compressing and fixing air bladder 51 is rapidly exhausted, and the measurement process is terminated.

The display process of step S308 may be carried out after the storage process of step S310. The blood pressure measurement is carried out in the pressurization process but may be carried out in the depressurization process.

(Calling and Displaying Process)

In the present embodiment, the blood pressure measurement data stored in the memory 39 is read out, and the read data is displayed on the display unit 40 in response to the instruction by the operation of the operation unit 41. This is hereinafter called the calling process. In the present embodiment, the switch 41D or 41E is operated to instruct the execution of the calling and displaying process.

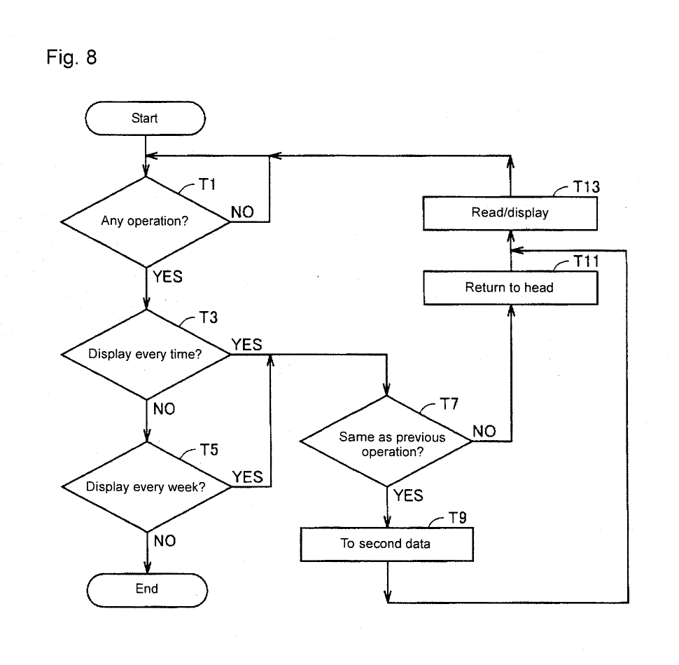

The calling process will be described with reference to FIG. 8. Assume that sufficient number of records R1, R2, and R3 are stored in ascending order of the values of the data DN and WN in the memory 39. Also assume that user A is specified by the operation of the switch 41B. A case in which the calling and displaying process of the record R1 or R2 is carried out for the blood pressure measurement data of the user A will be described. The process is similarly carried out when the switch 41B is operated to specify the user B.

First, the input determination unit 103 of the CPU 30 determines whether or not any of the switches of the operation unit 41 is operated based on the output signal from the operation unit 41 (step T1). If the input determination unit 103 determines that some kind of operation is performed (YES in step T1), the type of switch that was operated is determined based on the output signal from the operation unit 41 (steps T3, T5).

If determined that the switch 41E is operated as a result of the determination (YES in step T3), the process proceeds to the process of step T7 to be described later. If not determined that the switch 41E is operated (NO in step T3), whether or not the switch 41D is operated is determined (step T5). If determined that the switch 41D is operated, the process proceeds to step T7.

If determined that the operated switch is nether switch 41D nor 41E (NO in step T5), the series of calling and displaying process is terminated.

In step T7, whether or not the type of the operation switch for this time is the same as the type of the previously operated operation switch (step T7). The input determination unit 103 stores the data of the type of the operation switch determined based on the output signal from the operation unit 41. The switch type determined this time and the stored switch type are compared every time the operation is carried out, and whether or not the switch of the same type as the previous type is operated is determined based on the comparison result (step T7). If determined that the switch of the same type is operated (YES in step T7), the display processing unit 104 updates the value of each pointer P1, P2, and P3 so as to indicate the record stored next of the records R1, R2, and R3 indicated by the current value of the pointers P1, P2, and P3 in each region E2a, E3a, and E4a of the memory 39 (step T9). Each record indicated by the updated pointers P1, P2 and P3 is read out, the image data is generated based on each read record, and the generated image data is provided to the display unit 40 (step T13). The display unit 40 displays a screen according to the provided image data. The process thereafter proceeds to step T1.

If determined as not the same as the type of the operation switch of the previous time (NO in step T7), the value of each pointer P1, P2, and P3 is updated so as to indicate the records R2, R3, and R4 stored at the head of the corresponding regions E2a, E3a, and E4a (step T11). The process thereafter proceeds to step T13. In step T13, the data of each record indicated by the current pointer is read out, the image data is generated based on the read data, and the generated image data is provided to the display unit 40. The display unit 40 displays a screen according to the provided image data.

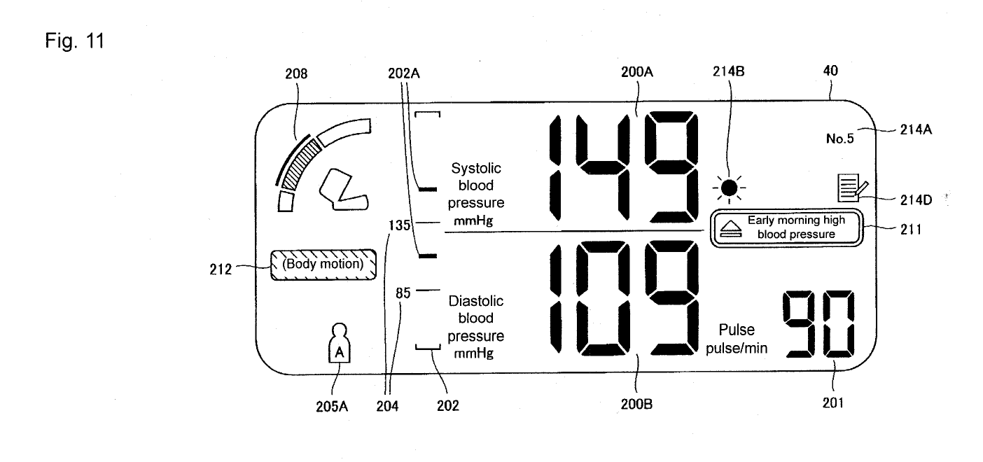

When the operation of the switch 41E is repeated, the every-time processing portion 113 of the display processing unit 104 operates to call and display the data of the record R1 indicated by the pointer P1 of the region E2a. Therefore, the record R1 of the blood pressure data is read out according to the time series order of the measurement time from the region E2a, and displayed every time the switch 41E is operated. One example of the display screen in this case is shown in FIG. 11.

Data 214D indicating the calling and displaying, and value 214A of the data DN of the record R1 that is called and displayed are displayed on the display screen of FIG. 11. Furthermore, if the every-time processing portion 113 determines that the data TM of the record R1 corresponds to the morning time, data 214B of sunshine mark indicating morning time is displayed. In FIG. 11, a segment 202A indicating the systolic blood pressure of the data 200A and the diastolic blood pressure of the data 200B displayed on the same screen is displayed in place of the display of the mark 203 assigned as a scale for sectionalizing into segments of every 10 mmHg and the display of the bar graph 215 in the blood pressure level bar 202. Therefore, the user who feels difficulty in reading with the display of the bar graph 215 can switch from the screen of FIG. 10 to the screen in which the bar graph 215 is not displayed as shown in FIG. 11. Other display items in the screen of FIG. 11 are the same as those described in FIG. 9 or FIG. 10.

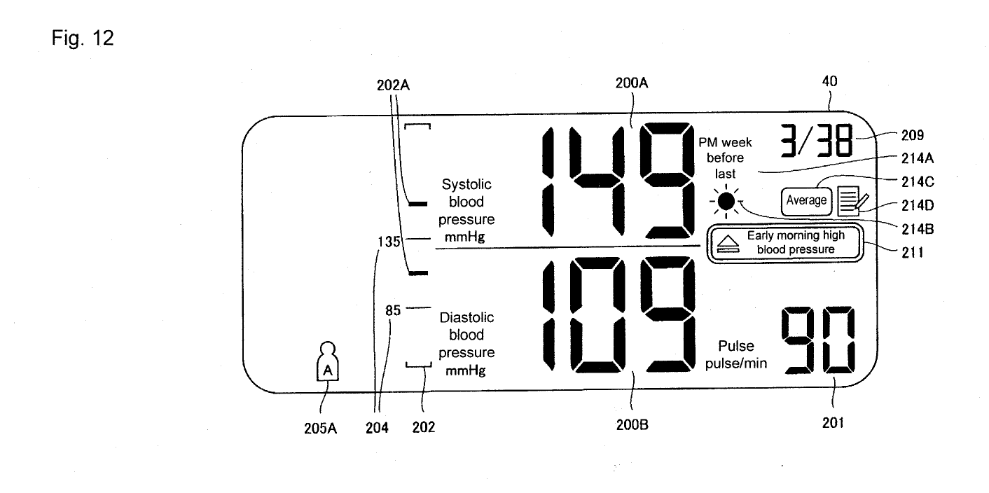

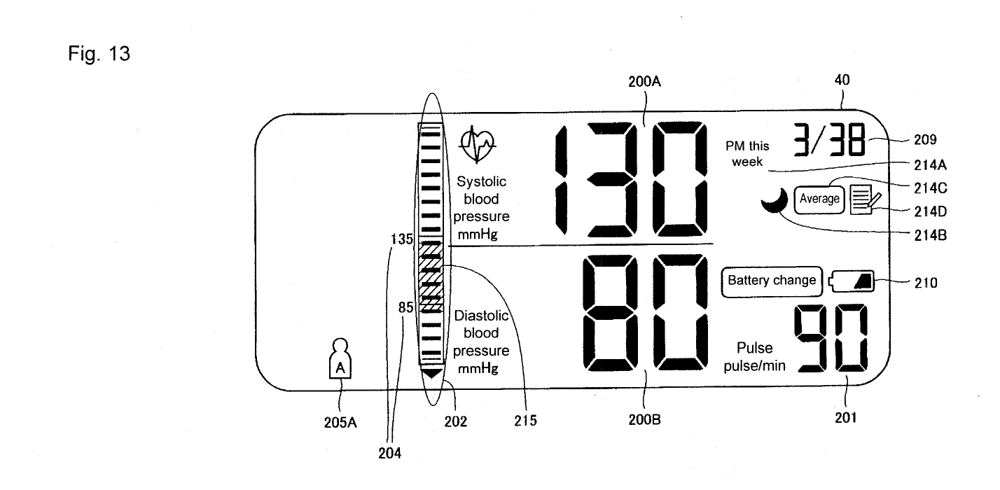

When the switch 411D is repeatedly operated, the data of the record R2 indicated by the pointer P2 of the region E3a is called and displayed for every operation. When the switch 412D is repeatedly operated, the data of the record R3 indicated by the pointer P3 of the region E4a is called and displayed for every operation. Therefore, the record R2 (R3) of the blood pressure data is read according to the time series order of the measurement time from the region E3a (E4a) and displayed. One example of such display is shown in FIG. 12 and FIG. 13.

FIG. 12 shows a display screen in a case where the record R2 is called and displayed. In FIG. 12, the character data 214A of the week before last is displayed based on the value indicated by the data WN of the record R2. The data 214B and 214C of a sunshine mark indicating that the displayed record R2 is the average data of the morning measurement time zone are displayed. Other display items are similar to those shown in FIG. 9 to FIG. 11.

FIG. 13 shows a display screen in a case where the record R3 is called and displayed. In FIG. 13, the character data 214A of this week is displayed based on the value indicated by the data WN of the record R3. The data 214B and 214C of a moon mark indicating that the displayed record R3 is the average data of the night measurement time zone are displayed. Other display items are similar to those shown in FIG. 9 to FIG. 12.

According to the screen display of the display unit 40 described above, the blood pressure measurement result of the systolic blood pressure value and the diastolic blood pressure value is digitally displayed by the data 200A and 200B in the same screen, and the bar graph 215 having the data 200A and 200B as upper and lower limit values is displayed in the blood pressure level bar 205 scaled and sectionalized by 10 mmHg using the mark 203, and hence the user can intuitively understand the level of the blood pressure value, which was difficult to understand with digital display, with the bar graph 215.

The reference value 204 is displayed in a numerical value close to the scale corresponding to the reference value 204 of the high blood pressure section of the scales of the blood pressure level bar 202, and hence whether or not the measured blood pressure corresponds to the high blood pressure can be readily checked.

As shown in FIG. 9, the bar of the bar graph 215 is displayed to extend (increase) and contract (decrease) in a step wise manner in cooperation with the change of the blood pressure data 200 detected in the pressurization process of the blood pressure measurement. Thus the person to be measured can intuitively grasp the progress status of pressurization.

The past blood pressure measurement results stored in the memory 39 are displayed as the digital numerical value and the bar graph 215 simultaneously on the same screen, as shown in FIGS. 10 to 13. Furthermore, the screen can be switched to display the continuous past history data according to the operation of the switch. Therefore, the transition state of the blood pressure value in units of daily or weekly can be determined in a glance with the digital numerical value and the bar graph 215 even if the display region is small.

(Other Blood Pressure Measurement Device)

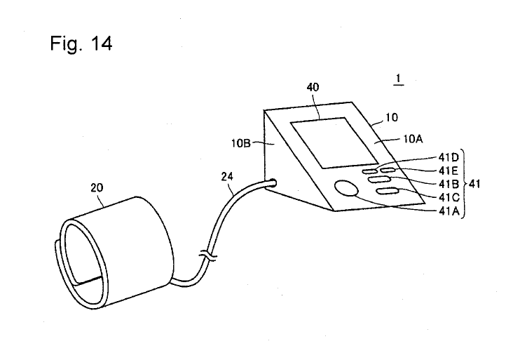

The blood pressure measurement device according to the present invention is not limited to an automatic winding type in which the main body and the cuff are integrally configured as shown in FIG. 1. For instance, the blood pressure measurement device in which the cuff 20 to be manually wrapped around the measurement site and the sphygmomanometer main body 10B are configured as separate bodies through the air tube 24, as shown in FIG. 14, may be adopted. The operation unit 41 and the display unit 40 are arranged on the front surface 10A of the housing of the sphygmomanometer main body 10B.

The embodiment disclosed herein is illustrative in all aspects and should not be construed as being restrictive. The technical scope of the invention is defined by the Claims and all modifications equivalent in meaning to the description of the Claims and within the scope of the invention are to be encompassed herein.

Although the present invention has been described and illustrated in detail, it is clearly understood that the same is by way of illustration and example only and is not to be taken by way of limitation, the scope of the present invention being interpreted by the terms of the appended claims.

What is claimed is:

1. A blood pressure measurement device comprising:
a cuff to be attached to a measurement site of a living body;
a control unit for calculating a blood pressure while adjusting pressure of the cuff for blood pressure measurement;
a storage unit for storing data of the blood pressure calculated by the control unit;
a display unit; and
a display operation unit operated to input an instruction related to display using the display unit; wherein
the blood pressure indicated by the blood pressure data read from the storage is displayed with a bar divided by a plurality of segments of a predetermined unit and simultaneously displayed with a numerical value in a same screen of the display unit based on the instruction input through the display operation unit.

2. The blood pressure measurement device according to claim 1, further comprising:
a display processing unit for reading out the blood pressure data from the storage unit and displaying the blood pressure indicated by the read blood pressure data with the bar and simultaneously displaying with the numerical value every time a predetermined instruction is input through the display operation unit; wherein
the blood pressure data to be read from the storage unit is read according to a time-series order of a measurement time every time the predetermined instruction is input.

3. The blood pressure measurement device according to claim 1, wherein the blood pressure data read from the storage unit indicates a systolic blood pressure and a diastolic blood pressure.

4. The blood pressure measurement device according to claim 3, wherein
the systolic blood pressure refers to an average of the systolic blood pressures of the blood pressure data measured within a predetermined period; and
the diastolic blood pressure refers to an average of the diastolic blood pressures of the blood pressure data measured within the predetermined period.

5. The blood pressure measurement device according to claim 3, wherein
the systolic blood pressure refers to an average of the systolic blood pressures of the blood pressure data for one week measured in a predetermined time band of one day; and
the diastolic blood pressure refers to an average of the diastolic blood pressures of the blood pressure data for one week measured in the predetermined time band.

6. The blood pressure measurement device according to claim 1, wherein a reference value indicating a predetermined blood pressure section is displayed in association at a position indicating a blood pressure value corresponding to the reference value on the bar.

7. The blood pressure measurement device according to claim 6, wherein the predetermined blood pressure section refers to a section of early morning high blood pressure.

8. The blood pressure measurement device according to claim 1, wherein the blood pressure that fluctuates according to the adjustment is displayed with the bar and simultaneously displayed with the numerical value in the same screen of the display unit in the process of adjusting the pressure of the cuff.
